(19) United States
(12) Patent Application Publication
(10) Pub. No.: US 2025/0213110 A1
(43) Pub. Date: Jul. 3, 2025

(54) SELECTIVE ACTIVATION DURING COHERENCE-BASED EYE TRACKING

(71) Applicant: Apple Inc., Cupertino, CA (US)

(72) Inventors: Thierry Oggier, Langnau am Albis (CH); Edward Vail, Menlo Park, CA (US)

(21) Appl. No.: 18/951,840

(22) Filed: Nov. 19, 2024

Related U.S. Application Data
(60) Provisional application No. 63/615,330, filed on Dec. 28, 2023.

(51) Int. Cl. A61B 3/113; A61B 3/10

(52) U.S. Cl. CPC A61B 3/113; A61B 3/102

(57) ABSTRACT

Various implementations disclosed herein include electronic devices, systems, and methods that determine a current position of a portion of an eye based on coherence-based measurements. An example electronic device may include a tracking component that includes an integrated circuit that includes a plurality of lasers and one or more photodiodes. An example method may include determining an expected position of a portion of an eye relative to the tracking component. The method may further include selectively activating a subset of the plurality of lasers to project light towards the portion of the eye, wherein reflected light of the projected light are sensed via a subset of the one or more photodiodes based on the expected position of the portion of the eye. The method may further include determining a current position of the portion of the eye based on coherence-based measurements using the projected light and the reflected light.

### CROSS-REFERENCE TO RELATED APPLICATIONS

This Application claims the benefit of U.S. Provisional Application Ser. No. 63/615,330 filed Dec. 28, 2023, which is incorporated herein in its entirety.

### TECHNICAL FIELD

The present disclosure generally relates to electronic devices, and in particular, to systems, methods, and devices for determining a current position of an eye of users of electronic devices based on coherence-based measurements.

### BACKGROUND

Some existing eye-tracking techniques analyze light reflected off front surfaces of the eye to estimate eye characteristics. For example, such techniques may estimate the user's gaze direction using multiple glints reflected off the front eye surface to identify locations along the user's gaze (e.g., pupil center, eye center, and cornea center). Other techniques use images of a retina to track eye characteristics. The robustness, accuracy, and/or efficiency of existing eye tracking techniques may be improved.

### SUMMARY

Various implementations disclosed herein include devices, systems, and methods that track a state of a user's eye (e.g., eye position/orientation, gaze direction accommodation, pupil dilation, etc.) using coherence-based measurement (e.g., optical coherence tomography (OCT)). The coherence-based measurement may provide sub-surface information, e.g., depth, cross section, or a volumetric model of the eye, based on reflections/scattering of light (e.g., typically using invisible light in the near-infrared spectrum) and provide efficiency, small size, and other benefits over existing techniques.

Some implementations disclosed herein include devices, systems, and methods that track gaze using coherence-based measurement that are achieved by (a) selectively activating subsets of less than all light sources based on current eye position and (b) integrating the light sources and photodiode array into an integrated circuit/chip (i.e., solid state circuit). In some implementations, the light sources may include lasers or integrated light sources embedded within or adjacent to the integrated circuit. In some implementations, the light sources may include lasers or monolithic silicon light sources embedded within or adjacent to the integrated circuit. The light sources may include vertical cavity surface-emitting lasers (VCSELs).

In some implementations, chip integration may be enabled, for example, based on VCSEL-on-Silicon (VoS) technology with integrated VCSELs and photodiodes. In some implementations, this may include photodiode integration within the VCSEL structure. In some implementations, this may involve front-side photodiode integration in complementary metal-oxide semiconductor (CMOS) application specific integrated circuit (ASIC) die, back-side photodiode integration, and/or using a shaped surface (e.g., bumps for every VCSEL).

In some implementations, the integrated OCT-based gaze tracking may reduce occlusion issues during transmission and receiving of reflected light from projected light, allow a smaller footprint on the integrated circuit (e.g., using modular camera attachment-enabled optical (MCO) device(s)), and a reduction in power consumption. The technique may use an integrated self-mixing interferometer (SMI) array. In some implementations, techniques described herein may project collimated spots in an eye box (e.g., when the eyeball is found within the eye box, select only VCSELs needed to cover the eyeball area). In some implementations, SMI switching may be used to switch between modes (e.g., extended coverage during an active mode or eye box scan mode, gaze tracking, sleep mode “wake-on-gaze”, and the like).

In general, another innovative aspect of the subject matter described in this specification can be embodied in methods at an electronic device having a processor and a tracking component. The tracking component includes an integrated circuit that includes a plurality of lasers and one or more photodiodes. The method may include the actions of determining, by the tracking component, an expected position of a portion of an eye relative to the tracking component, based on the expected position of the portion of the eye, selectively activating a subset of the plurality of lasers to project light towards the portion of the eye, where reflected light of the projected light are sensed via a subset of the one or more photodiodes, and determining a current position of the portion of the eye based on coherence-based measurements using the projected light and the reflected light.

These and other embodiments may each optionally include one or more of the following features.

In some aspects, determining the expected position of the portion of the eye relative to the tracking component is based on a previous tracked position of the eye. In some aspects, determining the expected position of the portion of the eye relative to the tracking component is based on sensor data received from one or more sensors, the sensor data corresponding to a plurality of reflections of light produced by the plurality of lasers or another light source and reflected from the eye.

In some aspects, the tracking component further comprises an integrated self-mixing interferometer (SMI) array. In some aspects, the tracking component is configured to selectively switch scanning modes based on an SMI switching technique of the SMI array.

In some aspects, determining the expected position of the portion of the eye relative to the tracking component is based on a first scanning mode, and the method further includes switching to a second scanning mode, different than the first scanning mode, to scan the eye based on the determined current position and an orientation of the eye.

In some aspects, selectively activating the subset of the plurality of lasers is based on a scanning mode of the tracking component.

In some aspects, the coherence-based measurement comprises optical coherence tomography (OCT). In some aspects, the coherence-based measurements comprises sub-surface information associated with an features below a surface of the eye.

In some aspects, the integrated circuit comprises a semiconductor material having a non-planar surface shape configured to direct the projected light from each of the plurality of lasers in a plurality of different directions.

In some aspects, at least one of the one or more photodiodes are integrated in a back-side of the tracking component. In some aspects, the one or more photodiodes are integrated in a front-side of the tracking component in a complementary metal-oxide semiconductor (CMOS) application specific integrated circuit (ASIC) die.

In some aspects, the one or more photodiodes are infrared (IR) photodiodes. In some aspects, the plurality of lasers are monolithic silicon light sources that are integrated on the integrated circuit. In some aspects, the plurality of lasers are vertical cavity surface-emitting lasers (VCSELs). In some aspects, the one or more photodiodes comprise a photodiode array. In some aspects, the projected light of the plurality of lasers comprises infrared (IR) light.

In some aspects, the tracking component comprises a processor positioned on the integrated circuit. In some aspects, the electronic device is a head-mounted device (HMD).

In accordance with some implementations, a non-transitory computer readable storage medium has stored therein instructions that are computer-executable to perform or cause performance of any of the methods described herein. In accordance with some implementations, a device includes one or more processors, a non-transitory memory, and one or more programs; the one or more programs are stored in the non-transitory memory and configured to be executed by the one or more processors and the one or more programs include instructions for performing or causing performance of any of the methods described herein.

### BRIEF DESCRIPTION OF THE DRAWINGS

So that the present disclosure can be understood by those of ordinary skill in the art, a more detailed description may be had by reference to aspects of some illustrative implementations, some of which are shown in the accompanying drawings.

FIG. 1 illustrates an environment in which extended reality (XR) content is provided to one or more users wearing head mounted displays (HMDs) in accordance with some implementations.

FIG. 2A illustrates an example of a user wearing an HMD with a tracking component in accordance with some implementations.

FIG. 2B illustrates an example view of a refractive/diffractive medium of the HMD of FIG. 2A in accordance with some implementations.

FIG. 2C illustrates use of the tracking component of the HMD of FIG. 2A in accordance with some implementations.

FIG. 3 illustrates an example eye-tracking component of FIG. 2A based on selectively activating lasers to project light towards an eye in accordance with some implementations.

FIGS. 4A and 4B illustrate an example eye-tracking system using coherence-based measurement in accordance with some implementations.

FIGS. 5A, 5B, and 5C illustrate different operating modes for an example eye-tracking system in accordance with some implementations.

FIG. 6 illustrates data from an exemplary coherence-based measurement of an eye based on the operating mode of FIG. 5A in accordance with some implementations.

FIG. 7 illustrates data from an exemplary coherence-based measurement of an eye based on the operating mode of FIG. 7B in accordance with some implementations.

FIG. 8 illustrates data from an exemplary coherence-based measurement of an eye based on the operating mode of FIG. 7C in accordance with some implementations.

FIG. 9 illustrates data from an exemplary coherence-based measurement of an eye in accordance with some implementations.

FIG. 10 is a flowchart representation of a method for determining a current position of the portion of the eye based on coherence-based measurements using the projected light and the reflected light in accordance with some implementations.

FIG. 11 illustrates device components of an exemplary device in accordance with some implementations.

FIG. 12 illustrates an example HMD in accordance with some implementations.

In accordance with common practice the various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DESCRIPTION

Numerous details are described in order to provide a thorough understanding of the example implementations shown in the drawings. However, the drawings merely show some example aspects of the present disclosure and are therefore not to be considered limiting. Those of ordinary skill in the art will appreciate that other effective aspects or variants do not include all of the specific details described herein. Moreover, well-known systems, methods, components, devices and circuits have not been described in exhaustive detail so as not to obscure more pertinent aspects of the example implementations described herein.

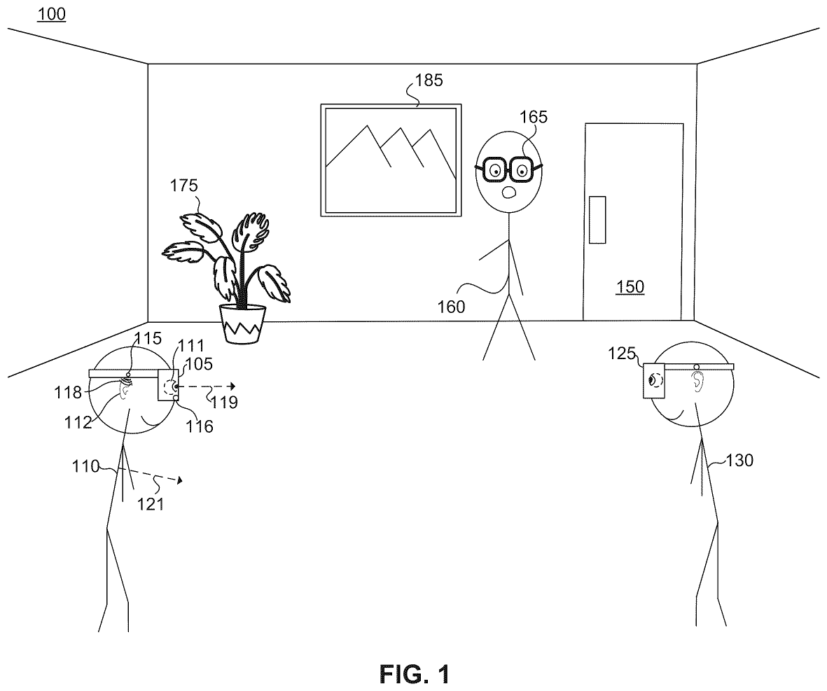

FIG. 1 illustrates a real-world physical environment 100 including a first user 110 wearing a first device 105, a second user 130 wearing a second device 125, a third user 160 wearing a third device 165, a wall-hung picture 185, a plant 175, and a door 150. In some implementations, one or more of the devices 105, 125, 165 is configured to provide content based on one or more sensors on the respective devices or to share information and/or sensor data with one another. In some implementations, one or more of the devices 105, 125, 165 provide content that provides augmentations in XR using sensor data. The sensor data may be used to understand that a user's state is associated with providing user assistance, e.g., a user's appearance or behavior or an understanding of the environment may be used to recognize a need or desire for assistance.

In the example of FIG. 1, the first device 105 includes one or more sensors 116 that capture light-intensity images, depth sensor images, audio data or other information about the user 110 and the physical environment 100. For example, the one or more sensors 116 may capture images of the user's forehead, eyebrows, eyes, eye lids, cheeks, nose, lips, chin, face, head, hands, wrists, arms, shoulders, torso, legs, or other body portion. Sensor data about a user's eye 111, as one example, may be indicative of various user characteristics, e.g., the user's gaze direction 119 over time, user saccadic behavior over time, user eye dilation behavior over time, etc. The one or more sensors 116 may capture audio information including the user's speech and other user-made sounds as well as sounds within the physical environment 100.

One or more sensors, such as one or more sensors 115 on device 105, may identify user information based on proximity or contact with a portion of the user 110. As example, the one or more sensors 115 may capture sensor data that may provide biological information relating to a user's cardiovascular state (e.g., pulse), body temperature, breathing rate, etc.

The one or more sensors 116 or the one or more sensors 115 may capture data from which a user orientation 121 within the physical environment can be determined. In this example, the user orientation 121 corresponds to a direction that a torso of the user 110 is facing.

In some implementations, the content provided by the device 105 and sensor features of device 105 may be provided using components, sensors, or software modules that are sufficiently small in size and efficient with respect to power consumption and usage to fit and otherwise be used in lightweight, battery-powered, wearable products such as wireless ear buds or other ear-mounted devices or head mounted devices (HMDs) such as smart and/or augmented reality (AR) glasses. Features can be facilitated using a combination of multiple devices. For example, a smart phone (connected wirelessly and interoperating with wearable device(s)) may provide computational resources, connections to cloud or internet services, location services, etc.

In some implementations, data is shared amongst a group of devices to improve user state or environment understanding. For example, device 125 may share information (e.g., images, audio, or other sensor data) corresponding to user 110 or the physical environment 100 (including information about user 130 or user 160) with device 105 so that device 105 can better understand user 110 and physical environment 100.

In some implementations, devices 105, 125, 165 are head mounted devices (HMDs) that present visual or audio content (e.g., extended reality XR content) or have sensors that obtain sensor data (e.g., visual data, sound data, depth data, ambient lighting data, etc.) about the environment 100 or sensor data (e.g., visual data, sound data, depth data, physiological data, etc.) about the users 110, 130, 160. Such information may, subject to user authorizations, permissions, and preferences, be shared amongst the device 105, 125, 165 to enhance the user's experiences on such devices.

In some implementations, the devices 105, 125, 165 obtain physiological data (e.g., EEG amplitude/frequency, pupil modulation, eye gaze saccades, etc.) from the users 110, 130, 160 via one or more sensors that are proximate or in contact with the respective user 110, 130, 160. For example, the device 105 may obtain pupillary data (e.g., eye gaze characteristic data) from an inward facing eye tracking sensor. In some implementations, the devices 105, 125, 165 include additional sensors for obtaining image or other sensor data of the physical environment 100.

In some implementations, the devices 105, 125, 165 are wearable devices such as ear-mounted speaker/microphone devices (e.g., headphones, ear pods, etc.), smart watches, smart bracelets, smart rings, smart/AR glasses, or other head-mounted devices (HMDs). In some implementations, the devices 105, 125, 165 are handheld electronic devices (e.g., smartphones or tablets). In some implementations, the devices 105, 125, 165 are laptop computers or desktop computers. In some implementations, the devices 105, 125, 165 have input devices such as audio command input systems, gesture recognition-based input systems, touchpads or touch-sensitive displays (also known as a “touch screen” or “touch screen display”). In some implementations, multiple devices are used together to provide various features. For example, a smart phone (connected wirelessly and interoperating with wearable device(s)) may provide computational resources, connections to cloud or internet services, location services, etc.

FIG. 1 illustrates an example in which the devices within the physical environment 100 include HMD devices 105, 125, 165. Numerous other types of devices may be used including mobile devices, tablet devices, wearable devices, hand-held devices, personal assistant devices, AI-assistant-based devices, smart speakers, desktop computing devices, menu devices, cash register devices, vending machine devices, juke box devices, or numerous other devices capable of presenting content, capturing sensor data, or communicating with other devices within a system, e.g., via wireless communication. For example, assistance may be provided to a vision impaired person to help the person understand a menu by providing data from the menu to a device being worn by the vision impaired person, e.g., enabling that device to enhance the user's understanding of the menu by providing visual annotations, audible cues, etc.

In some implementations, the devices 105, 125, 165 include eye tracking systems for detecting eye position, gaze direction, and eye movements. For example, an eye tracking system may include one or more infrared (IR) light-emitting diodes (LEDs), an eye tracking camera (e.g., near-IR (NIR) camera), and an illumination source (e.g., an NIR light source) that emits light (e.g., NIR light) towards the eyes of the user. Moreover, an illumination source on a device may emit NIR light to illuminate the eyes of the user and the NIR camera may capture images of the eyes of the user. In some implementations, images captured by the eye tracking system may be analyzed to detect position and movements of the eyes of the user, or to detect other information about the eyes such as pupil dilation or pupil diameter. Moreover, the point of gaze estimated from the eye tracking system may enable gaze-based interaction with, e.g., content shown on the device or with the real world environment. Additional cameras may be included to capture other areas of the user (e.g., an HMD with a jaw cam to view the user's mouth, a down cam to view the body, an eye cam for tissue around the eye, and the like). These cameras and other sensors can detect motion of the body, or signals of the face modulated by the breathing of the user (e.g., remote PPG).

In some implementations, the devices 105, 125, 165 have graphical user interfaces (GUIs), one or more processors, memory and one or more modules, programs or sets of instructions stored in the memory for performing multiple functions. In some implementations, the users 110, 130, 160 may interact with a GUI through voice commands, finger contacts on a touch-sensitive surface, hand/body gestures, remote control devices, or other user input mechanisms. In some implementations, the functions include viewing/listening to content, image editing, drawing, presenting, word processing, website creating, disk authoring, spreadsheet making, game playing, telephoning, video conferencing, e-mailing, instant messaging, workout support, digital photographing, digital videoing, web browsing, digital music playing, or digital video playing. Executable instructions for performing these functions may be included in a computer readable storage medium or other computer program product configured for execution by one or more processors.

In some implementations, the devices 105, 125, 165 employ various physiological or behavioral sensor, detection, or measurement systems. Detected physiological data may include, but is not limited to, EEG, electrocardiography (ECG), electromyography (EMG), functional near infrared spectroscopy signal (fNIRS), blood pressure, skin conductance, or pupillary response. Detected behavioral data may include, but is not limited to, facial gestures, facial expressions, body gestures, or body language based on image data, voice recognition based on acquired audio signals, etc.

In some implementations, the devices 105, 125, 165 (or other devices) may be communicatively coupled to one or more additional sensors. For example, a sensor (e.g., an EDA sensor) may be communicatively coupled to a device 105, 125, 165 via a wired or wireless connection, and such a sensor may be located on the skin of a user (e.g., on the arm, placed on the hand/fingers of the user, etc.). For example, such a sensor can be utilized for detecting EDA (e.g., skin conductance), heart rate, or other physiological data that utilizes contact with the skin of a user. Moreover, a device 105, 125, 165 (using one or more sensors) may concurrently detect multiple forms of physiological data in order to benefit from synchronous acquisition of physiological data or behavioral data. Moreover, in some implementations, the physiological data or behavioral data represents involuntary data, e.g., responses that are not under conscious control. For example, a pupillary response may represent an involuntary movement. In some implementations, a sensor is placed on the skin as part of a watch device, such as a smart watch.

In some implementations, one or both eyes of a user, including one or both pupils of the user present physiological data in the form of a pupillary response (e.g., eye gaze characteristic data). The pupillary response of the user may result in a varying of the size or diameter of the pupil, via the optic and oculomotor cranial nerve. For example, the pupillary response may include a constriction response (miosis), e.g., a narrowing of the pupil, or a dilation response (mydriasis), e.g., a widening of the pupil. In some implementations, a device may detect patterns of physiological data representing a time-varying pupil diameter. In some implementations, the device may further determine the interpupillary distance (IPD) between a right eye and a left eye of the user.

The user data (e.g., upper facial feature characteristic data, lower facial feature characteristic data, and eye gaze characteristic data, etc.), including information about the position, location, motion, pose, etc., of the head or body of the user, may vary in time and a device 105, 125, 165 (or other devices) may use the user's data to track a user state. In some implementations, the user data includes texture data of the facial features such as eyebrow movement, chin movement, nose movement, cheek movement, etc. For example, when a person (e.g., user 110, 130, 160) performs a facial expression or micro expression associated with lack of familiarity or confusion, the upper and lower facial features can include a plethora of muscle movements that are used to assess the state of the user based on the captured data from sensors.

The physiological data (e.g., eye data, head/body data, etc.) and behavioral data (e.g., voice, facial recognition, etc.) may vary in time and the device may use the physiological data or behavioral data to measure a physiological/behavioral response or the user's attention to object or intention to perform an action. Such information may be used to identify a state of the user with respect to whether the user needs or desires assistance.

Information about such assistance predictions and how a user's own data is used may be provided to a user and the user given the option to opt out of automatic predictions/use of their own data and given the option to manually override assistance features. In some implementations, the system is configured to ensure that users' privacy is protected by requiring permissions to be granted before user state is assessed or assistance is enabled.

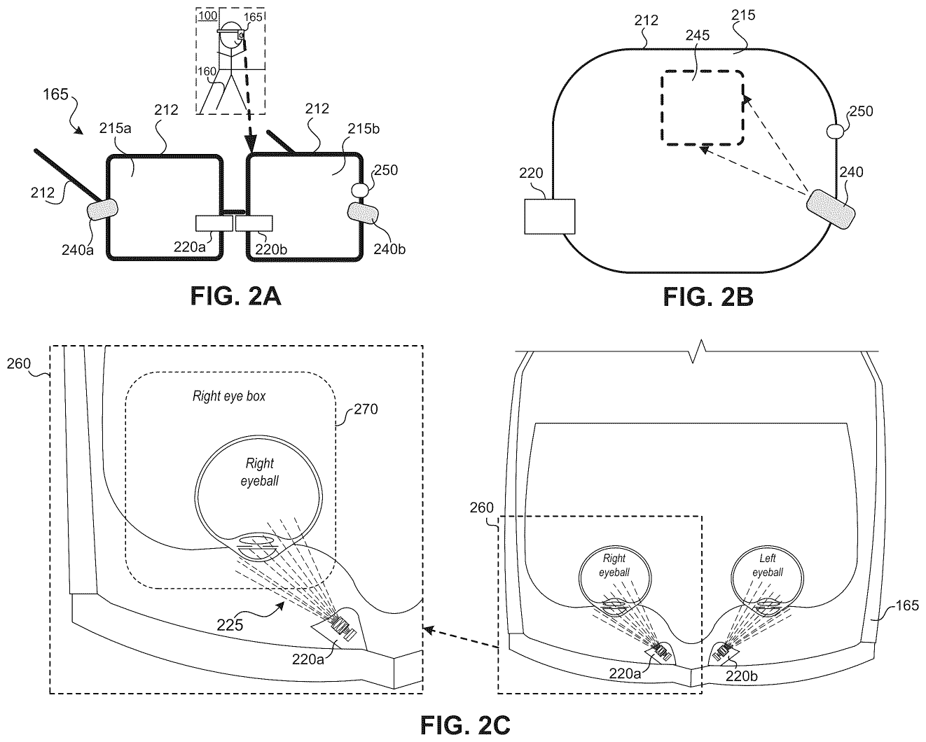

FIG. 2A illustrates an example of a user wearing an HMD in accordance with some implementations. In particular, FIG. 2A illustrates an example operating environment of the real-world environment 100 (e.g., a room) from FIG. 1, including the user 160 wearing device 165 (e.g., an HMD). In this example, the device 165 is an HMD that includes a transparent or a translucent display that includes a medium through which light representative of images is directed to the eyes of user 160. In particular, device 165 is an HMD that may also be referred to herein as smart glasses, “AR glasses” or “XR glasses.” Such XR glasses may include a transparent display to view the physical environment and be provided a display to view other content via retinal projection technology that projects graphical images within a view of a person's retina or onto a person's retina.

As illustrated, device 165 includes a frame 212 that can be worn on the user's head and may include additional extensions (e.g., arms) that are placed over ears of the user 160 to hold the frame in place on the user's head. The device 165 includes two displays for a left eye and a right eye of the user 160. The frame 212 supports a first lens 215a, and a second lens 215b. Each lens 215 includes a refractive/diffractive medium. Each lens 215 may be configured as a stack that includes a bias (+/−) for prescription lenses, a waveguide for projecting a display and/or housing or embedding components such as a plurality of IR light sources and transparent conductors, and the like.

The device 165 further includes a tracking component 220a and tracking component 220b, for each lens 215a, 215b, respectively. The tracking component 220 may include an integrated circuit that includes a plurality of light sources such as lasers (e.g., VCSELs, monolithic silicon light sources, etc.) and one or more photodiodes (e.g., a photodiode array). A processor associated with the tracking component 220 may be used to selectively activate a subset of the plurality of light sources, and determine a current position of a portion of the eye based on coherence-based measurements using the projected light and the reflected light. Additional hardware and configuration information and usage implementations of the tracking component 220 is further described herein.

In some implementations, the device 165 further includes projector 240a, 240b, for each lens 215a, 215b, respectively. A projector 240 may be used to display XR content to the user (e.g., virtual content that appears to the user at some focal point distance away from the device 165 based on the configuration of the lens). A waveguide stacked within the lens 215 may be configured to bend and/or combine light that is directed toward the eye of the user 160 to provide the appearance of virtual content within the real physical environment 100. In some implementations, the device 165 may only include one projector 240. For example, a pair of XR glasses for a user that only displays XR content on one side of the device 165 so the user 160 is less distracted and can have a greater view of the physical environment 100.

In some implementations, the device 165 further includes a controller 250. For example, the controller 250 may include a processor and a power source that controls the light being emitted from the tracking component 220. In some implementations, the controller 250 may be a microcontroller that can control the processes described herein for assessing characteristics of the eye (e.g., gaze direction, eye orientation, identifying an iris of the eye) based on the sensor data obtained from the photodiodes. Alternatively, the controller 250 may be communicatively coupled (e.g., wireless communication) with another device, such as a mobile phone, tablet, and the like, and the controller may send data collected from the photodiodes to be analyzed by the other device. In the exemplary implementation, the device 165 (with the controller 250) is a stand-alone unit that can project the virtual content via projector 240 and assess characteristics of the eye via light sources for eye tracking purposes without communicating with another device. In some implementations, the scanning tracking component 220 and the plurality of photodiodes are individually addressable. For example, a processor within the controller 250 can manage the tracking component 220 and determine a current position of a portion of the eye based on coherence-based measurements using the projected light and the reflected light obtained from the photodiodes.

FIG. 2B illustrates an example view of a refractive/diffractive medium (e.g., lens 215) of the HMD 165 of FIG. 2A in accordance with some implementations. In particular, FIG. 2B illustrates a refractive/diffractive medium with components (some transparent/translucent) for an eye tracking system and XR display for the device 165. In this example, the lens 215 includes a tracking component 220, a projector 240, a display 545, and a controller 250. The controller 250 may control and provide power to each component via transparent conductors. The transparent conductors may be configured to have a size that is small enough and/or are made of one or more transparent materials (e.g., transparent conducting films (TCFs)) so as to not be detectable by a human eye, and thus would be considered transparent and/or translucent when viewing content through the lens 215. Transparent conductors (e.g., connections between each component) may include an optically transparent and electrically conductive material including, but not limited to, indium tin oxide (ITO), wider-spectrum transparent conductive oxides (TCOs), conductive polymers, metal grids and random metallic networks, carbon nanotubes (CNT), graphene, nanowire meshes, and/or ultra thin films. In some implementations, transparent conductors may include semi-transparent conductor materials such as silver nano traces or the like. For example, semi-transparent material may refer to a material that is not necessarily transparent but thin enough that the material is not perceptible to a human eye.

The XR display system of the device 165 through lens 215 includes a projector 240 and a display 245 that may appear to the user as illustrated at the location of display 245. However, the light projected from the projector 240, as powered and controlled by the controller 250, is not directly projected as illustrated. Instead, the light from projector 240 may be bent, via a waveguide, such that the XR content being displayed at display 245 appears to the user 160 at some focal point distance away from the device 165 based on the configuration of the waveguide.

In some implementations, the device 165 may only have one of the lens' 215 display XR content (e.g., the left eye lens 215b would be a normal lens without a tracking component 220b, without a projector 240, and thus without a display 545). For example, a left eye view would only present pass-through content of the physical environment 100 (e.g., such as a normal pair of glasses), and the right eye view would have both pass through content of the physical environment 100, and have the capability to present XR content to the right lens 215a only. For example, only the right lens 215a would include a projector 240, a display 245 to present the XR content, and a tracking component 220 to track the right eye movement (e.g., as the eye may be gazing towards the XR content on the display 245).

FIG. 2C illustrates use of the tracking component 220 of the HMD of FIG. 2A in accordance with some implementations. In particular, FIG. 2C illustrates a transverse (horizontal) plane for a cross-sectional top-down view of a portion of the user's head that includes the left eye and the right eye while the user 160 is wearing the device 165. Additionally, the view provides a cross-sectional top-down view of the tracking components 220a,b. The expanded portion of area 260 provides an expanded view of the right eyeball of the user 160 and the right side tracking component 220a. Additionally, the expanded portion of area 260 provides a view of the projected light rays 225 towards the right eyeball and a right eye box 270. The right eye box 270 illustrates a potential volume of space that the eyeball could potentially be during use of the device 165. In an exemplary implementation, ideally, the tracking components 220a,b cover all eye boxes across population. As further discussed herein, an eye box, such as right eye box 270, may be determined during an initial scan of an individual user (e.g., an eye box scan mode), so the system can determine the outer limits for the eye tracking system (e.g., no need to utilize resources and scan outside of the individual's eye box).

The tracking component 220 may be a coherence-based tracking system and will be further discussed herein. In an exemplary implementation, the coherence-based tracking system may include one or more coherence-based measurement devices and a controller. The coherence-based measurement device may include a wave source that direct waves (e.g., light) toward the eye of the user at least some of which penetrate the eye's front surfaces and into interior portions of the eye structures and reflects or is scattered by interior aspects of the eye structures. The reflected and/or scattered waves may be detected by the coherence-based measurement device (e.g., a sensor). Based on the reflections/scatterings, the controller can determine an eye characteristic of the user, including but not limited to, a gaze direction, accommodation state, pupil dilation state, etc. Thus, in various implementations, the waves are emitted by the one or more wave sources, reflected or are scattered off portions of the eye of the user, and are detected by one or more sensors for use in assessing eye characteristics.

In some implementations, the coherence-based measurement device is an optical coherence tomography (OCT) device. The coherence-based measurement device may produce waves (e.g., light) that are directed towards the eye of the user, splitting off a portion of the waves for coherence measurement purposes. The coherence-based measurement device may use multiple wavelengths that penetrate the tissue of the eye to varying extents before being reflected or scattered. The reflections or scatterings of the waves are sensed by the coherence-based measurement device by interfering them with the portion of the waves that were split off. A Fourier transform of the wavelength dependent signal that is received to produce data that represents 3D volumetric information, a depth profile, or cross-section of a portion of the eye. Areas of the eye that are similar to water, e.g., interior portions of the eye structures, may reflect or scatter few waves versus areas of the eye, such as the front and back edges of the cornea, which may reflect or scatter relatively more of the waves. In some OCT implementations, the coherence-based measurement device may produce variable or swept frequencies to produce reflections/scatterings at different depths within the tissue. The reflection/scattering of the waves at different depths may thus provide information about the composition of the internal structures of the eye. Accordingly, the coherence-based measurement device may be used to produce data from which a 3D volumetric structure, depth characteristics, and other such attributes can be determined.

A display may emit light in a first wavelength range and the coherence-based measurement device may emit light in a second wavelength range. Similarly, the coherence-based measurement device may detect light in the second wavelength range. In various implementations, the first wavelength range is a visible wavelength range (e.g., a wavelength range within the visible spectrum of approximately 400-700 nm) and the second wavelength range is a near-infrared wavelength range (e.g., a wavelength range within the near-infrared spectrum of approximately 700-1400 nm). In one example, the coherence-based measurement device is configured to produce infrared light centered at a wavelength of about 850 nm.

In various implementations, detected eye characteristics are used to enable user interaction. For example, a detected gaze direction may be used to control a user interface (e.g., the user selects an option on the display by looking at it), provide foveated rendering (e.g., present a higher resolution in an area of the display the user is looking at and a lower resolution elsewhere on the display), or reduce geometric distortion (e.g., in 3D rendering of objects on the display). Similarly, a detected accommodative state (or user intention to focus at a particular distance), may be used to determine which content a user intends to look at and adjust the user experience accordingly, e.g., by enhancing or changing that content, etc. In another example, a detected pupil dilation state is used to assess a user response to (e.g., interest in) content presented on the display.

In some implementations, the coherence-based measurements provide range measurements based on the interference between a projected wave and the same wave reflected/scattered back. Such measurements may be used to creates 3D volumetric scans of the eye, based on measuring through tissues and providing information about the eye structures including their thickness. For example, thickness information may be provided with less than 10 μm accuracy. In some implementations, the coherence-based measurements include scanning performed in multiple directions and/or using multiple techniques. For example, the scanning may include an A-scan, along a Z/depth axis, and/or a B-scan, along an X axis and a Y axis. A B-scan may be performed using a micromirror (e.g., a MEMS mirror). In some implementations, a scan, such as an A-scan, is performed by moving a reference mirror.

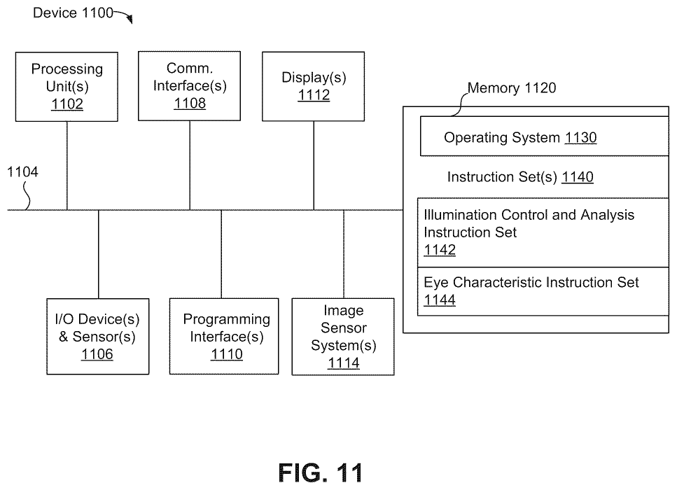

Some implementations disclosed herein track eye characteristics (e.g., eye position and/or orientation in 6 degrees of freedom, gaze direction, eye accommodative state, pupil dilation state, etc.) based on information provided by a coherence-based measurement of the eye, e.g., using optical coherence tomography (OCT). For example, this may involve creating a 3D model of a user's eye (e.g., once in a lifetime or infrequently), performing a tracking scan using coherence-based measurement of the eye, and determining the eye characteristic based on the scan and the 3D model. An example of such a technique is discussed below with reference to FIG. 11. In some implementations, the current position and/or orientation of the eye is determined by aligning (e.g., registering) the tracking scan data with the 3D model. In some implementations, the state of a portion of the eye (e.g., eye muscles such as the ciliary muscles or other portions such as the pupil opening) are assessed by comparing the tracking scan with the 3D model. An example of detecting accommodation state of the eye is discussed next.

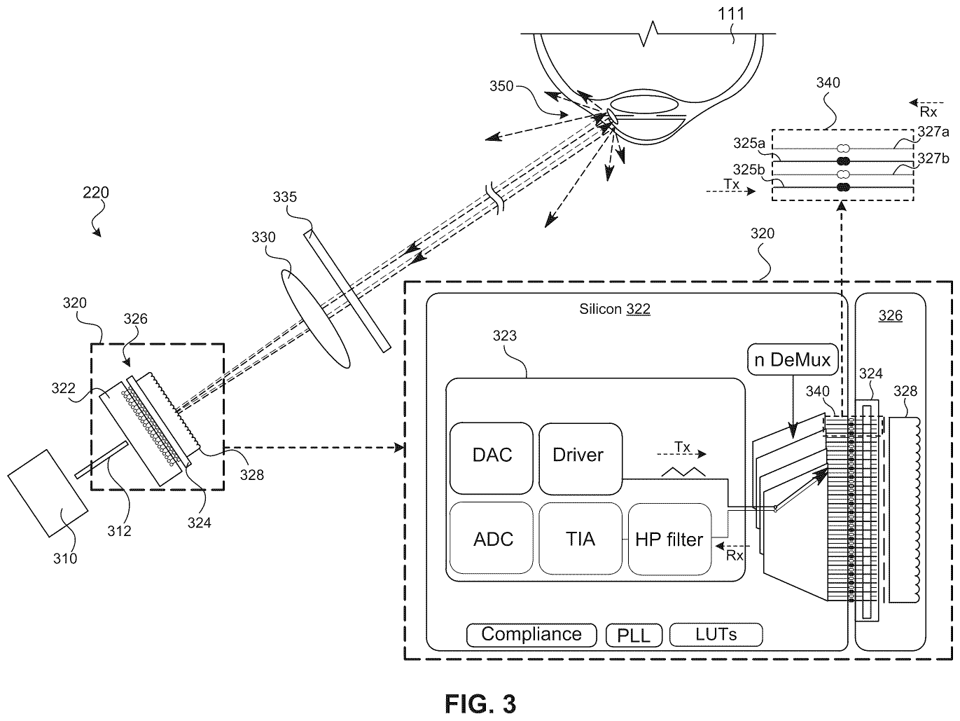

FIG. 3 illustrates an example eye-tracking component of FIG. 2A based on selectively activating lasers to project light towards an eye in accordance with some implementations. For example, FIG. 3 illustrates the structure of the tracking component 220 while projecting light (e.g., IR) towards a portion of the eye 111 and receiving reflected light (e.g., reflected light rays 350) from the projected light upon the eye 111.

In some implementations, the tracking component 220 includes a processor 310, an interface 312 to interface the processor and the self-mixing interferometer (SMI) array system, an SMI array architecture on an integrated circuit as shown in area 320, a collimating lens 330, and a cover glass 335. The SMI array architecture may include silicon 322, a buffer layer 324, SMI array 326, and a lens 328 (e.g., an “on-chip” lens). The buffer layer 324 may include a gallium arsenide (GaAs) layer, or another type of band gap semiconductor material.

The expanded portion of the area 320 provides further structural components of the tracking component 220. For example, in the silicon 322 portion, the tracking component 220 may further include several different processing components for sending (Tx) and receiving (Rx) the plurality of projected/reflected light signals. In particular, a processing portion 323 may include a digital-to-analog converter (DAC) and a driver to transmit one or more light signals via a multichannel data distributor, known more commonly as the demultiplexer or “Demux” which will send the light signals to the SMI array 326 through the buffer layer 324, where the SMI array 326 will transmit the light signals to the eye 111 through the lens 328. The processing portion 323 may further include a high pass (HP) band filter, a trans-impedance amplifier (TIA) chip, and an analog-to-digital converter (ADC) to process the received (Rx) reflected light signals from the demultiplexer (e.g., as received as projected light from the eye 111 from the SMI array 326 via the lens 328, buffer layer 324, etc.). The processing portion 323 may further include other processing components such as a compliance unit, a phase lock loop control system (PLL), and look-up tables (LUTs) for DAC and/or ADC look up values.

As illustrated in blow up area 340, the tracking component 220 includes a plurality of light sources (e.g., lasers, VSCELs, etc.) that transmit light signals 325a, 325b, etc., and the tracking component 220 includes a plurality of photodiodes (e.g., IR photodiodes) that receive a plurality of reflected light signals 327a, 327b, etc. The transmitted light signals 325 may be actively selected using one or more techniques further described herein. The plurality of light sources can be either integrated in the silicon 322 or the SMI array 326.

In some implementations, the photodiodes may be a part of the GaAs VCSEL structure, embedded in the silicon underneath, or a combination thereof. For example, an integrated self-mixing interferometer (SMI) structure may include photodiode detector integration within a VCSEL structure. In some implementations, an SMI structure may be included with a VCSEL-on-Silicon (VoS) device with a photodiode integrated in complementary metal-oxide semiconductor (CMOS) application specific integrated circuit (ASIC) die. In some implementations, a photodiode may be integrated as a front-side photodiode integrated in the CMOS ASIC die. Additionally, or alternatively, in some implementations, an SMI structure may be included with a VoS device as a back-side photodiode integrated in an additional backside illuminated sensor (BSI) layer (e.g., an IR BSI wafer layer, or the like).

In some implementations, an optical path for the sending/receiving of light signals in an exemplary SMI structure may be optimized and/or modified based on one or more components of the SMI structure (e.g., thickness of each die layer, layout/structure of the optical path, grating components, etc.). In some implementations, a GaAs die layer of the exemplary SMI structures may include a μ-optical bump on a backside of the GaAs die layer. For example, the μ-optical bump may be a non-planar surface shape (e.g., a bump) configured to direct the projected light in different directions. In some implementations, the μ-optical bump may be included for every VCSEL for beam steering capabilities.

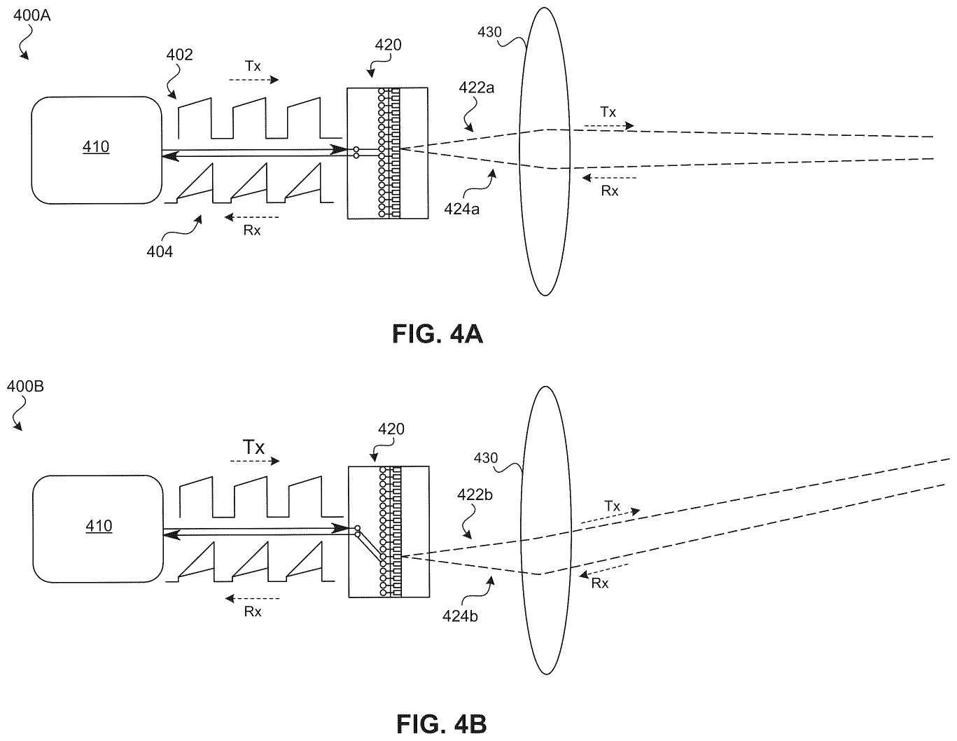

FIG. 4A and FIG. 4B illustrate example environments 400A and 400B, respectively, of an eye-tracking system using coherence-based measurements in accordance with some implementations. In particular, FIGS. 4A and 4B illustrate Tx/Rx waveforms and corresponding light characteristics associated with selectively activating two different channels of an SMI array 420 for projecting a light signal to an eye and receiving a reflected eye signal. For example, the integrated SMI architecture includes a controller 410 that transmits shaped pulses 402 to an SMI array 420 that transmits a light signal through a specified SMI channel to generate an output light signal 422a,b via a light source (e.g., a laser such as a VSCEL) that is sent to an object (e.g., an eye) via a collimating lens 430. Then, in response to a light reflected off of the object, a reflected light signal 424a,b is received at the SMI array 420 via the collimating lens 430. The reflected light signal 424a,b is then detected by a photodiode of the specified SMI channel and received by the controller 410 as OCT pulses 404. In some implementations, the switching between the different SMI channels such as selecting a different VSCEL or photodiode, may be accomplished using an electrical switch in silicon.

In some implementations, based on the OCT pulses 404, the controller 410 may determine a current position of a portion of the eye based on coherence-based measurements using the reflected light. Additionally, or alternatively, in some implementations, based on the OCT pulses 404, the controller 410 may utilize an eye tracking system algorithm to determine which SMI beams hit the eyeball and be able to classify the SMI light beams into beams that hit different portions of the eye (e.g., skin, cornea, sclera, lens, etc., or a combination thereof) based on the reflected light signals 424 based on a distinctive signature along its captured range.

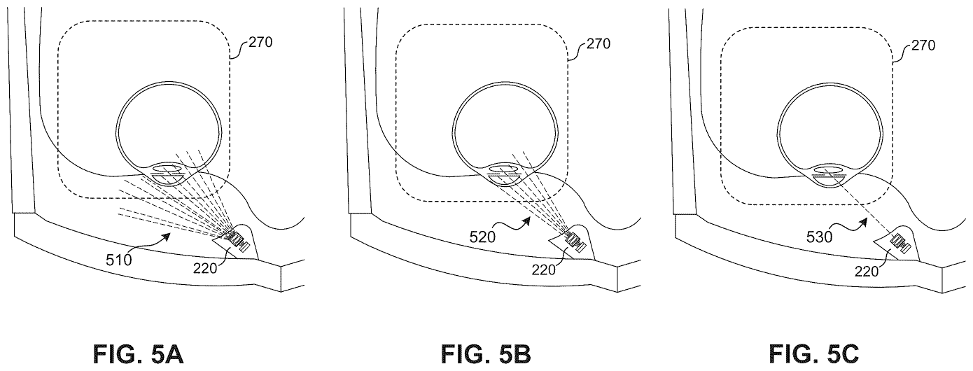

FIGS. 5A, 5B, and 5C illustrate different operating modes for an example eye-tracking system in accordance with some implementations. In particular, FIGS. 5A-5C illustrate a similar view as the expanded portion of area 260 of FIG. 2C, which includes a transverse (horizontal) plane for a cross-sectional top-down view of a portion of the user's head that includes the right eye while the user 160 is wearing the device 165.

FIG. 5A illustrates a first mode for extended coverage for detecting an eye and an area around the eye. For example, as illustrated, the tracking component 220 disseminates a wide range of projected light signals 510 towards the eye. Based on the extended coverage of the first mode, the tracking component 220 can determine the individual eye box 270. Thus, the first operating mode of the tracking component 220 may be referred to as eye box scan mode as an initial scanning procedure. In some implementations, the purpose of the first mode (e.g., active mode or eye box scan mode) is to find the individual user eye box 270 within the field of view of the SMI array. In some implementations, this first mode may only be needed at a beginning of an eye tracking session. In some implementations, because of slippage of the HMD or glasses (e.g., device 105) and other changes with the eye tracking system, the active mode or eye box scan mode may be needed as required as an overall scan periodically or whenever a change is detected (e.g., recalibration the eye tracking system). In the eye box scan mode, typically all SMIs (e.g., projected light signals 510) are captured (e.g., obtaining all receive signals (Rx) from the photodiode array).

FIG. 5B illustrates a second mode for minimum coverage for tracking an eye. For example, as illustrated, the tracking component 220 disseminates a narrow range of projected light signals 520 towards the eye, but enough light signals to cover the main portions of the eye. Based on the minimum coverage of the second mode, the tracking component 220 can track the gaze of the user. Thus, the second operating mode of the tracking component 220 may be referred to as a gaze tracking mode. In some implementations, the purpose of the second mode is to track the eye movements within the field of view of the SMI array. For example, after the active mode or eye box scanning mode is complete, the gaze tracker system via the tracking component 220 knows where the eyeball is and only needs to utilize those SMIs that hit the eyeball as illustrated in FIG. 5B (e.g., projected light signals 520). In some implementations, the gaze tracking mode allows to reduce either power by running less SMIs or the system can be sped up by running those selected SMIs faster. In some implementations of the gaze tracking mode, after the position of the eye and gaze is detected, the eye ball may be checked for movements only with a minimum number of SMI, and as soon as movement is detected, all SMI needed for the gaze tracking mode are turned on until the eye ball is still again.

FIG. 5C illustrates a third mode for monitoring an eye. For example, as illustrated, the tracking component 220 disseminates a single projected light signal 530 towards the eye. Based on the single projected light signal 530, the tracking component 220 can monitor whether the eye moves periodically. Thus, the third operating mode of the tracking component 220 may be referred to as a sleep mode. In some implementations, the purpose of the third mode is to save power when there is not a need for eye tracking. For example, depending on a measured OCT scan of a single or a minimal number of SMIs (e.g., projected light signal 530), the system can be kept in a “sleep” mode or woken up. In some implementations, the sleep mode may have to be tuned to use as little as possible numbers of SMI with as long of a frame and/or sample time as possible to reduce power consumption. In some implementations, gaze angle may only roughly be estimated, but accurate enough to wake up the eye tracking system given a certain constellation.

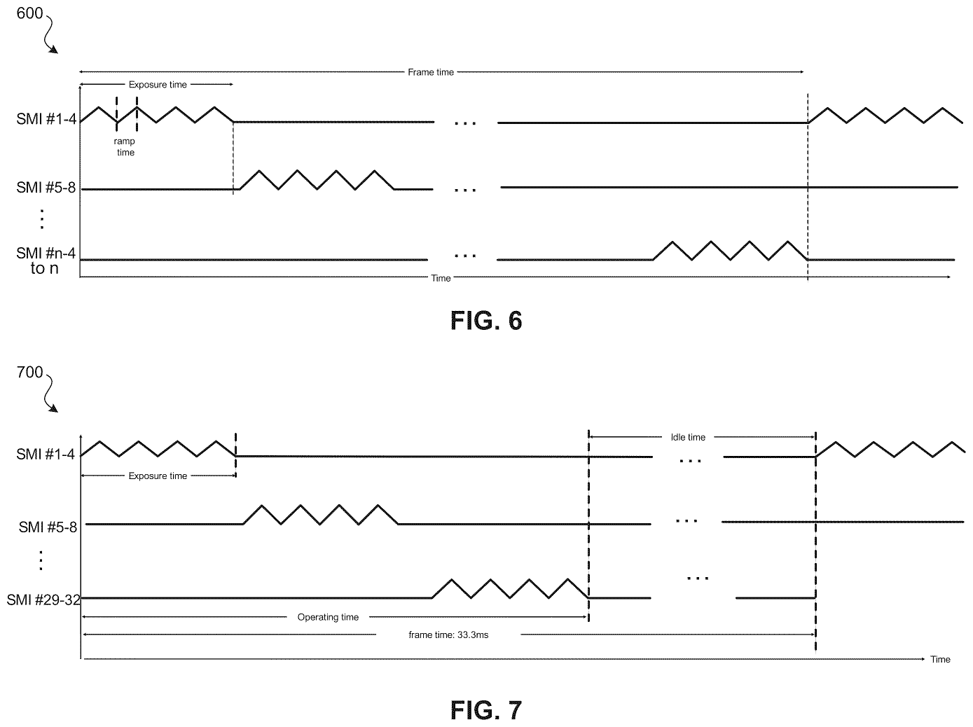

FIG. 6 illustrates data as graph 600 from an exemplary coherence-based measurement of an eye based on the operating mode of FIG. 5A in accordance with some implementations. In particular, graph 600 illustrates an active mode (also referred to as an eye box scan mode) as discussed herein with reference to FIG. 5A. For example, graph 600 illustrates obtaining light signals from a set of photodiodes associated with an SMI array (e.g., OCT pulses) as measured by frequency on the y-axis, and time on the x-axis.

In some implementations, the purpose of the active mode or eye box scan mode is to find the individual eye box within the field of view of the SMI array. In some implementations, this active mode may only be needed at a beginning of an eye tracking session. In some implementations, because of slippage of the HMD or glasses (e.g., device 105) and other changes with the eye tracking system, the active mode or eye box scan mode may be needed as required as an overall scan periodically or whenever a change is detected. In the eye box scan mode, typically all SMIs are captured (e.g., obtaining all receive signals (Rx) from the photodiode array).

In some implementations, as illustrated by graph 600, the OCT gaze tracking system may handle four SMIs in parallel, and then always run groups of four SMIs in bundles and then switching to the next four SMIs until either the entire eye box has been scanned (e.g., all SMIs captured) or the location of the eyeball has been identified. For example, SMIs #1-4 are captured together for a first exposure time period, then SMIs #5-8 are captured together for a second exposure time period, and then the next bundle of four SMIs continuously until the number n of SMIs are captured. The total number n of SMIs may vary from 10 to 1000s of SMIs dependent on the space available and the accuracy needed.

FIG. 7 illustrates data as graph 700 from an exemplary coherence-based measurement of an eye based on the operating mode of FIG. 5B in accordance with some implementations. In particular, graph 700 illustrates a gaze tracking mode as discussed herein with reference to FIG. 5B. For example, graph 700 illustrates obtaining light signals from a set of photodiodes associated with an SMI array (e.g., OCT pulses) as measured by frequency on the y-axis, and time on the x-axis.

In some implementations, the purpose of the gaze tracking mode is to track the eye movements within the field of view of the SMI array. For example, after the active mode or eye box scanning mode is complete, the gaze tracker system knows where the eyeball is and only needs to utilize those SMIs that hit the eyeball as illustrated in FIG. 5B. In some implementations, the gaze tracking mode allows to reduce either power by running less SMIs or the system can be sped up by running those selected SMIs faster. In the specific example as illustrated by graph 800, the eye tracking system is running 32 SMIs of all the integrated SMI array (e.g., running 32 SMIs of a 100 or more SMIs in total).

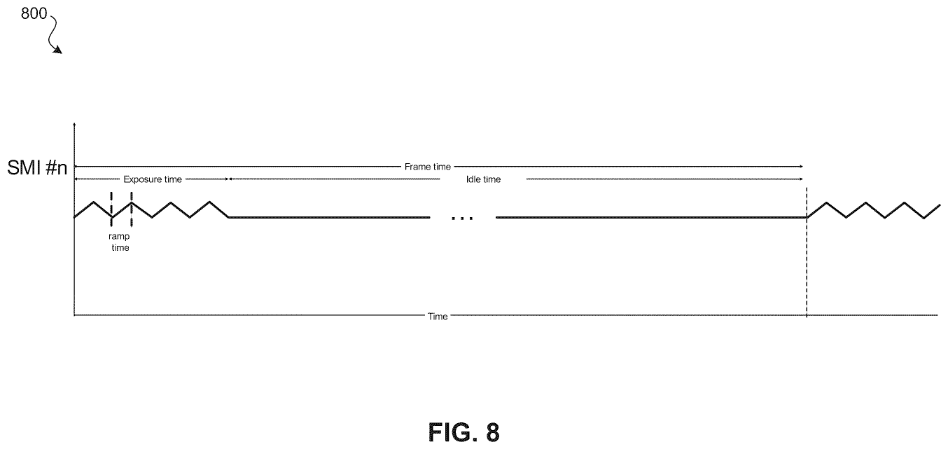

FIG. 8 illustrates data as graph 800 from an exemplary coherence-based measurement of an eye based on the operating mode of FIG. 5C in accordance with some implementations. In particular, graph 800 illustrates a sleeping mode as discussed herein with reference to FIG. 5C. For example, graph 800 illustrates obtaining a light signal from one photodiode associated with an SMI array (e.g., OCT pulses) as measured by frequency on the y-axis, and time on the x-axis.

In some implementations, the purpose of the sleep mode is to save power when there is not a need for eye tracking. For example, depending on a measured OCT scan of a single or a minimal number of SMIs, the system can be kept in a “sleep” mode or woken up. In some implementations, the sleep mode may have to be tuned to use as little as possible numbers of SMI with as long of a frame and/or sample time as possible to reduce power consumption. In some implementations, gaze angle may only roughly be estimated, but accurate enough to wake up the eye tracking system given a certain constellation.

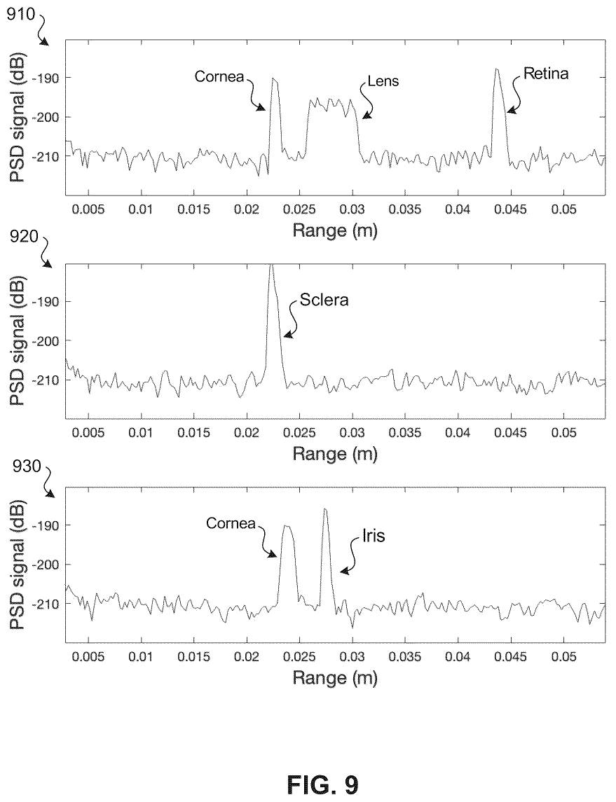

FIG. 9 illustrates data from an exemplary coherence-based measurement of an eye in accordance with some implementations. In particular, each graph 910, 920, and 930 provide a graphical representation of three different SMI beams as measured by an intensity on y-axis measured by a power spectral density (PSD) signal in decibels (dB) and a depth on the x-axis as measured by a range in meters (m). A unique scan signature of the tissue of the eye may be determined based on the intensity and depth measurements. For example, graph 910 illustrates the cornea, lens, and retina, graph 920 illustrates the sclera, and graph 930 illustrates the cornea and iris.

In some implementations, the process of transitioning from the active mode (e.g., eye box scan mode) to the gaze tracking mode, the eye tracking system algorithm needs to determine which SMI beams hit the eyeball. In an exemplary implementation of the eye tracking system algorithm, each determined SMI beam that hit the eye area (e.g., sclera, cornea, lid/skin, lens, etc.) have a distinctive signature along its captured range. Thus, as each SMI beam is directed to the eye, the unique scan signature of the tissue of the eye may be used to classify the SMI light beams into beams that hit skin, cornea, sclera, lens, etc., or a combination thereof. This classification may enable fast and accurate localization of the eyeball location.

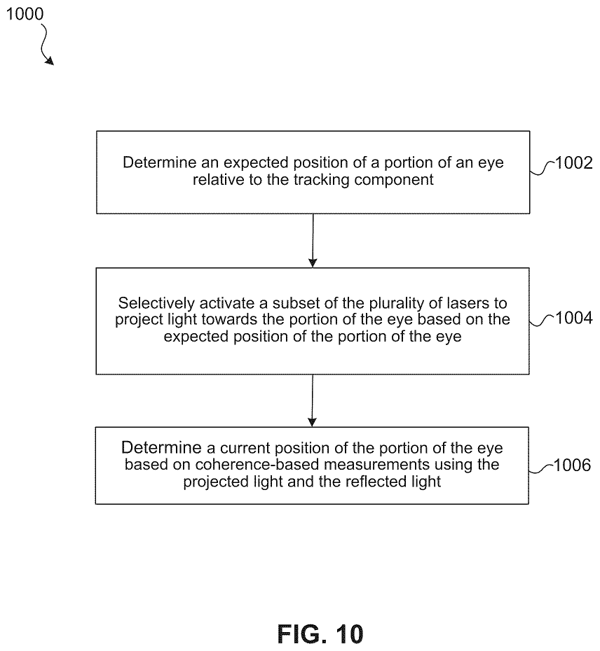

FIG. 10 is a flowchart illustrating an exemplary method 1000. In some implementations, a device (e.g., device 105) performs the techniques of method 1000 to determine a current position of an eye based on coherence-based measurements. In some implementations, the techniques of method 1000 are performed on a mobile device, desktop, laptop, HMD, or server device. In some implementations, the method 1000 is performed on processing logic, including hardware, firmware, software, or a combination thereof. In some implementations, the method 1000 is performed on a processor executing code stored in a non-transitory computer-readable medium (e.g., a memory). In some implementations, the method 1000 is performed in combination of one or more devices as described herein. For example, sensor data from a plurality of light sensors may be acquired at an HMD (e.g., device 105), but the processing of the data (e.g., assess an eye characteristic) may be performed at a separate device (e.g., a mobile device). In some implementations, the eye tracking system described herein maybe on a device that includes a display or a display system. Alternatively, in some implementations, the eye tracking system described herein maybe on a device that does not include a display, and only includes the gaze tracking system (e.g., a device located on or near the temple of a user).

In an exemplary implementation, the method 1000 is executed at an electronic device (e.g., an HMD, AR glasses, etc.) that includes a tracking component (e.g., tracking component 220). In some implementations, the tracking component includes an integrated circuit that includes a plurality of lasers (and one or more photodiodes (e.g., an SMI array that includes a photodiode array). In some implementations, the plurality of lasers are vertical cavity surface-emitting lasers (VCSELs), such as an SMI array that includes an array of VCSELs. In some implementations, the tracking component comprises a processor positioned on an integrated chip. In some implementations, the plurality of lasers are monolithic silicon light sources that are integrated on the integrated circuit.

In some implementations, the tracking component further includes an integrated self-mixing interferometer (SMI) array. In some implementations, the tracking component is configured to selectively switch scanning modes based on an SMI switching technique of the SMI array.

In some implementations, at least one of the one or more photodiodes are integrated in a back-side of the tracking component. In some implementations, the one or more photodiodes are integrated in a front-side of the tracking component in a complementary metal-oxide semiconductor (CMOS) application specific integrated circuit (ASIC) die. In some implementations, the one or more photodiodes comprise a photodiode array.

In some implementations, the one or more photodiodes are infrared (IR) photodiodes. In some implementations, the projected light of the plurality of lasers comprises infrared (IR) light.

In some implementations, the integrated circuit comprises a semiconductor material having a non-planar surface shape (e.g., bumps) configured to direct the projected light from each of the plurality of lasers in a plurality of different directions. For example, a bump (e.g., μ-optical bump 532 of FIGS. 5A and 5B) may be included for every VCSEL for beam steering capabilities.

At block 1002, the method 1000 determines an expected position of a portion of an eye relative to the tracking component. For example, determining a prior eyeball position before and/or during movement when initiating an eye tracking process.

In some implementations, determining the expected position of the portion of the eye relative to the tracking component is based on a previous tracked position of the eye. In other words, the expected position of the portion of the eye may be based on the eye's last tracked position or otherwise based on sensor data captured regarding the eye. For example, selective use of activation of VSCELs based on a previous eye scan.

In some implementations, determining the expected position of the portion of the eye relative to the tracking component is based on sensor data received from one or more sensors, the sensor data corresponding to a plurality of reflections of light produced by the plurality of lasers or another light source and reflected from the eye. For example, selective use of activation VSCELs based on other sensor data, such as other a scanning light source, or other projection component such as a micro-electromechanical system (MEMS) laser scanner, and the like. For example, a scanning light source produces glints (e.g., a specular reflection) by producing light that reflects off a portion of an eye. In some implementations, a glint may be a specular glint. In some implementations, if a scanning light source is used both for illuminating specular and diffusive parts of the object (e.g., eye 111 of the user 110), the specular “glints” must be in saturation in order to detect the diffusive area of the object. For example, a light source (e.g., a projection component such as a MEMS laser scanner, and the like) is flashed at an eye 111, and the detectors (e.g., photodiodes) detect the glints such as the reflected light rays from the eye 111.

In some implementations, determining the expected position of the portion of the eye relative to the tracking component is based on a first scanning mode. In some implementations, the method 1000 further includes switching to a second scanning mode, different than the first scanning mode, to scan the eye based on the determined current position and an orientation of the eye. For example, SMI switching may be used to switch between modes for extended coverage, gaze “active” tracking, sleep mode “wake-on-gaze”, etc.

At block 1004, the method 1000 selectively activate a subset of the plurality of lasers to project light towards the portion of the eye based on the expected position of the portion of the eye. For example, SMI switching may be used to activate/deactivate each of the SMIs individually, or in subsets (e.g., bundles of four SMIs as discussed herein).

In some implementations, selectively activating the subset of the plurality of lasers is based on a scanning mode of the tracking component. For example, as illustrated in FIGS. 5A-5C, different operating modes may be used depending on the detected scenario (e.g., active mode for extended coverage or an initial scan, gaze tracking when tracking eye movement, sleep mode to save power, etc.).

At block 1006, the method 1000 determines a current position of the portion of the eye based on coherence-based measurements using the projected light and the reflected light. For example, the eye tracking system discussed herein tracks gaze using coherence-based measurement (e.g., optical coherence tomography (OCT)). In some implementations, the coherence-based measurements includes sub-surface information associated with an features below a surface of the eye. For example, OCT may provide sub-surface information of the eye or portions of the eye.

In some implementations, the electronic receives sensor data from a set of one or more photodiodes, the sensor data corresponding to a plurality of reflections of light produced by the scanning light source and reflected from the eye. For example, the photodiodes (e.g., IR photodiodes) may be a sensor/detector that receives the reflections of light off of the eye (e.g., glints), such as reflections from the light rays 252, as illustrated in FIG. 3. In some implementations, the photodiodes may be positioned in an area in front of the lens, outside of the lens (e.g., on a frame of a device), in an area behind the lens, or embedded within the lens.

In some implementations, the method 1000 further includes determining a characteristic of the eye based on sensor data. For example, based on the sensor data obtained from the set of photodiodes, the eye tracking system described herein may be able to identify and/or track a position and/or orientation of an eye, a gaze direction, the cornea shape, and the like. In some implementations, determining the characteristic of the eye is based on determining at least one of a phase, an intensity, an angle, a timing, and a polarization of the light. In some implementations, determining the characteristic of the eye based on the sensor data includes determining a position of a pupil of the eye. For example, an XYZ coordinate in a 3D space may be determined for the pupil position based on the sensor data of the set of photodiodes. In some implementations, determining the characteristic of the eye based on the sensor data includes determining a gaze direction based on a detected reflection angle. In some implementations, determining the characteristic of the eye based on the sensor data includes determining a shape of the eye. For example, the eye tracking system described herein can determine pupil position (e.g., X,Y,Z coordinates) as well as gaze and other features such as eyelid, eyebrows, etc., and their associated movements.

In some implementations, determining an eye characteristic may be based on a determined location of the glint. For example, the eye characteristic may include a gaze direction, eye orientation, identifying an iris of the eye, or the like, for an eye-tracking system. For example, if the electronic device is an HMD, the eye-tracking system for the HMD can track gaze direction, eye orientation, identification of the iris, etc., of a user.

In some implementations, determining an orientation of the eye is based on identifying a pattern of the glints/light reflections in an image. In one example, gaze direction may be determined using the sensor data to identify two points on the eye, e.g., a cornea center and an eyeball center. In another example, gaze direction may be determined using the sensor data (e.g., a pattern of glints) to directly predict the gaze direction. For example, a machine learning model may be trained to directly predict the gaze direction based on the sensor data.

In some implementations, for iris identification, the user may be uniquely identified from a registration process or prior iris evaluation. For example, the method 1000 may include assessing the characteristic from the eye by performing an authentication process. The authentication process may include identifying an iris of an eye. For example, matching a pattern of glints/light reflections in an image with a unique pattern associated with the user. In some embodiments, the iris identification techniques (e.g., matching patterns), may be used for anti-spoofing. For example, there could be multiple enrolled patterns that may be changed and can be used to authenticate a user's iris against a pre-enrolled biometric template, and confirm that the user is the right person, a real person, and is authenticating in real-time. Iris identification may be used as a primary authentication mode or as part of a multi-factor or step-up authentication. The matching patterns may be stored in a database located on the HMD (e.g., device 105), another device communicatively coupled to the HMD (e.g., a mobile device in electronic communication with the HMD), an external device or server (e.g., connected through a network), or a combination of these or other devices.

FIG. 11 is a block diagram of an example device 1100. Device 1100 illustrates an exemplary device system configuration for a device (e.g., devices 105, 125, 165, etc.). While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the device 1100 includes one or more processing units 1102 (e.g., microprocessors, ASICs, FPGAs, GPUs, CPUs, processing cores, and/or the like), one or more input/output (I/O) devices and sensors 1106, one or more communication interfaces 1108 (e.g., USB, FIREWIRE, THUNDERBOLT, IEEE 802.3x, IEEE 802.11x, IEEE 802.16x, GSM, CDMA, TDMA, GPS, IR, BLUETOOTH, ZIGBEE, SPI, I2C, and/or the like type interface), one or more programming (e.g., I/O) interfaces 1110, one or more displays 1112, one or more interior and/or exterior facing image sensor systems 1114, a memory 1120, and one or more communication buses 1104 for interconnecting these and various other components.

In some implementations, the one or more communication buses 1104 include circuitry that interconnects and controls communications between system components. In some implementations, the one or more I/O devices and sensors 1106 include at least one of an inertial measurement unit (IMU), an accelerometer, a magnetometer, a gyroscope, a thermometer, one or more physiological sensors (e.g., blood pressure monitor, heart rate monitor, blood oxygen sensor, blood glucose sensor, etc.), one or more microphones, one or more speakers, a haptics engine, one or more depth sensors (e.g., a structured light, a time-of-flight, or the like), and/or the like.

In some implementations, the one or more displays 1112 are configured to present a view of a physical environment or a graphical environment to the user. In some implementations, the one or more displays 1112 correspond to holographic, digital light processing (DLP), liquid-crystal display (LCD), liquid-crystal on silicon (LCoS), organic light-emitting field-effect transitory (OLET), organic light-emitting diode (OLED), surface-conduction electron-emitter display (SED), field-emission display (FED), quantum-dot light-emitting diode (QD-LED), micro-electromechanical system (MEMS), and/or the like display types. In some implementations, the one or more displays 1112 correspond to diffractive, reflective, polarized, holographic, etc. waveguide displays. In one example, the device 105 includes a single display. In another example, the device 105 includes a display for each eye of the user.

In some implementations, the one or more image sensor systems 1114 are configured to obtain image data that corresponds to at least a portion of the physical environment. For example, the one or more image sensor systems 1114 include one or more RGB cameras (e.g., with a complimentary metal-oxide-semiconductor (CMOS) image sensor or a charge-coupled device (CCD) image sensor), monochrome cameras, IR cameras, depth cameras, event-based cameras, and/or the like. In various implementations, the one or more image sensor systems 1114 further include illumination sources that emit light, such as a flash. In various implementations, the one or more image sensor systems 1114 further include an on-camera image signal processor (ISP) configured to execute a plurality of processing operations on the image data.

The memory 1120 includes high-speed random-access memory, such as DRAM, SRAM, DDR RAM, or other random-access solid-state memory devices. In some implementations, the memory 1120 includes non-volatile memory, such as one or more magnetic disk storage devices, optical disk storage devices, flash memory devices, or other non-volatile solid-state storage devices. The memory 1120 optionally includes one or more storage devices remotely located from the one or more processing units 1102. The memory 1120 includes a non-transitory computer readable storage medium.

In some implementations, the memory 1120 or the non-transitory computer readable storage medium of the memory 1120 stores an optional operating system 1130 and one or more instruction set(s) 1140. The operating system 1130 includes procedures for handling various basic system services and for performing hardware dependent tasks. In some implementations, the instruction set(s) 1140 include executable software defined by binary information stored in the form of electrical charge. In some implementations, the instruction set(s) 1140 are software that is executable by the one or more processing units 1102 to carry out one or more of the techniques described herein.

The instruction set(s) 1140 include an illumination analysis instruction set 1142 and an eye characteristic instruction set 1144. The instruction set(s) 1140 may be embodied a single software executable or multiple software executables.

In some implementations, the illumination analysis instruction set 1142 is executable by the processing unit(s) 1102 to produce a reflection by directing light towards an eye using a scanning light source (e.g., a MEMS scanner), receive sensor data from a sensor (e.g., a set of one or more photodiodes) and determine a reflective property (e.g., a spectral property) of the reflection based on the sensor data. To these ends, in various implementations, the instruction includes instructions and/or logic therefor, and heuristics and metadata therefor.

In some implementations, the eye characteristic instruction set 1144 is executable by the processing unit(s) 1102 to determine a characteristic of the eye based on the sensor data such as identifying and tracking a position and/or orientation of an eye, a gaze direction, the cornea shape, and the like, using one or more of the techniques discussed herein or as otherwise may be appropriate. To these ends, in various implementations, the instruction includes instructions and/or logic therefor, and heuristics and metadata therefor.

Although the instruction set(s) 1140 are shown as residing on a single device, it should be understood that in other implementations, any combination of the elements may be located in separate computing devices. Moreover, FIG. 11 is intended more as functional description of the various features which are present in a particular implementation as opposed to a structural schematic of the implementations described herein. As recognized by those of ordinary skill in the art, items shown separately could be combined and some items could be separated. The actual number of instructions sets and how features are allocated among them may vary from one implementation to another and may depend in part on the particular combination of hardware, software, and/or firmware chosen for a particular implementation.

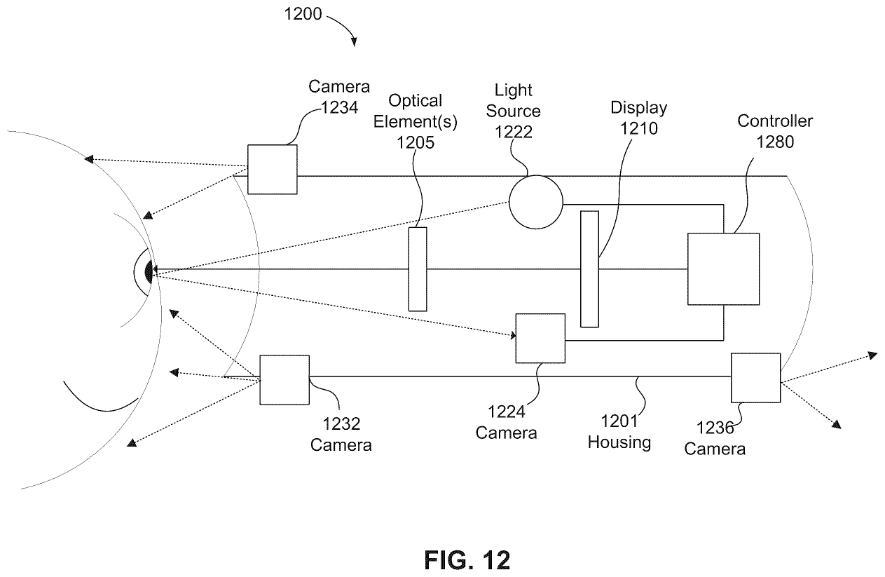

FIG. 12 illustrates a block diagram of an exemplary head-mounted device 1200 in accordance with some implementations. The head-mounted device 1200 includes a housing 1201 (or enclosure) that houses various components of the head-mounted device 1200. The housing 1201 includes (or is coupled to) an eye pad (not shown) disposed at a proximal (to the user) end of the housing 1201. In various implementations, the eye pad is a plastic or rubber piece that comfortably and snugly keeps the head-mounted device 1200 in the proper position on the face of the user 110 (e.g., surrounding the eye of the user).

The housing 1201 houses a display 1210 that displays an image, emitting light towards or onto the pupil of an eye of a user. In various implementations, the display 1210 emits the light through an eyepiece having one or more optical elements 1205 that refracts the light emitted by the display 1210, making the display appear to the user to be at a virtual distance farther than the actual distance from the eye to the display 1210. For example, optical element(s) 1205 may include one or more lenses, a waveguide, other diffraction optical elements (DOE), and the like. For the user to be able to focus on the display 1210, in various implementations, the virtual distance is at least greater than a minimum focal distance of the eye (e.g., 7 cm). Further, in order to provide a better user experience, in various implementations, the virtual distance is greater than 1 meter.

The housing 1201 also houses a tracking system including one or more light sources 1222, camera 1224, camera 1232, camera 1234, camera 1236, and a controller 1280. The one or more light sources 1222 emit light onto the eye of the user that reflects as a light pattern (e.g., a circle of glints) that can be detected by the camera 1224. Based on the light pattern, the controller 1280 can determine an eye tracking characteristic of the user. For example, the controller 1280 can determine a gaze direction or a blinking state (eyes open or eyes closed) of the user. As another example, the controller 1280 can determine a pupil center, a pupil size, or a point of regard associated with the pupil. Thus, in various implementations, the light is emitted by the one or more light sources 1222, reflects off the eye of the user, and is detected by the camera 1224. In various implementations, the light from the eye of the user is reflected off a hot mirror or passed through an eyepiece before reaching the camera 1224.

The display 1210 emits light in a first wavelength range and the one or more light sources 1222 emit light in a second wavelength range. Similarly, the camera 1224 detects light in the second wavelength range. In various implementations, the first wavelength range is a visible wavelength range (e.g., a wavelength range within the visible spectrum of approximately 400-700 nm) and the second wavelength range is a near-infrared wavelength range (e.g., a wavelength range within the near-infrared spectrum of approximately 700-1400 nm).

In various implementations, eye tracking (or, in particular, a determined gaze direction) is used to enable user interaction (e.g., the user selects an option on the display 1210 by looking at it), provide foveated rendering (e.g., present a higher resolution in an area of the display 1210 the user is looking at and a lower resolution elsewhere on the display 1210), or correct distortions (e.g., for images to be provided on the display 1210).

In various implementations, the one or more light sources 1222 emit light towards the eye of the user, which reflects in the form of a plurality of glints.

In various implementations, the camera 1224 is a frame/shutter-based camera that, at a particular point in time or multiple points in time at a frame rate, generates an image of the eye of the user. Each image includes a matrix of pixel values corresponding to pixels of the image which correspond to locations of a matrix of light sensors of the camera. In implementations, each image is used to measure or track pupil dilation by measuring a change of the pixel intensities associated with one or both of a user's pupils.

In various implementations, the camera 1232, camera 1234, and camera 1236 are frame/shutter-based cameras that, at a particular point in time or multiple points in time at a frame rate, can generate an image of the face of the user 110 or capture an external physical environment. For example, camera 1232 captures images of the user's face below the eyes, camera 1234 captures images of the user's face above the eyes, and camera 1236 captures the external environment of the user (e.g., environment 100 of FIG. 1). The images captured by camera 1232, camera 1234, and camera 1236 may include light intensity images (e.g., RGB) or depth image data (e.g., Time-of-Flight, infrared, etc.).

A physical environment refers to a physical world that people can sense or interact with without aid of electronic devices. The physical environment may include physical features such as a physical surface or a physical object. For example, the physical environment corresponds to a physical park that includes physical trees, physical buildings, and physical people. People can directly sense or interact with the physical environment such as through sight, touch, hearing, taste, and smell. In contrast, an extended reality (XR) environment refers to a wholly or partially simulated environment that people sense or interact with via an electronic device. For example, the XR environment may include augmented reality (AR) content, mixed reality (MR) content, virtual reality (VR) content, or the like. With an XR system, a subset of a person's physical motions, or representations thereof, are tracked, and, in response, one or more characteristics of one or more virtual objects simulated in the XR environment are adjusted in a manner that comports with at least one law of physics. As one example, the XR system may detect head movement and, in response, adjust graphical content and an acoustic field presented to the person in a manner similar to how such views and sounds would change in a physical environment. As another example, the XR system may detect movement of the electronic device presenting the XR environment (e.g., a mobile phone, a tablet, a laptop, or the like) and, in response, adjust graphical content and an acoustic field presented to the person in a manner similar to how such views and sounds would change in a physical environment. In some situations (e.g., for accessibility reasons), the XR system may adjust characteristic(s) of graphical content in the XR environment in response to representations of physical motions (e.g., vocal commands).

There are many different types of electronic systems that enable a person to sense or interact with various XR environments. Examples include head mountable systems, projection-based systems, heads-up displays (HUDs), vehicle windshields having integrated display capability, windows having integrated display capability, displays formed as lenses designed to be placed on a person's eyes (e.g., similar to contact lenses), headphones/earphones, speaker arrays, input systems (e.g., wearable or handheld controllers with or without haptic feedback), smartphones, tablets, and desktop/laptop computers. A head mountable system may have one or more speaker(s) and an integrated opaque display. Alternatively, a head mountable system may be configured to accept an external opaque display (e.g., a smartphone). The head mountable system may incorporate one or more imaging sensors to capture images or video of the physical environment, or one or more microphones to capture audio of the physical environment. Rather than an opaque display, a head mountable system may have a transparent or translucent display. The transparent or translucent display may have a medium through which light representative of images is directed to a person's eyes. The display may utilize digital light projection, OLEDs, LEDs, uLEDs, liquid crystal on silicon, laser scanning light source, or any combination of these technologies. The medium may be an optical waveguide, a hologram medium, an optical combiner, an optical reflector, or any combination thereof. In some implementations, the transparent or translucent display may be configured to become opaque selectively. Projection-based systems may employ retinal projection technology that projects graphical images onto a person's retina. Projection systems also may be configured to project virtual objects into the physical environment, for example, as a hologram or on a physical surface.

It will be appreciated that the implementations described above are cited by way of example, and that the present invention is not limited to what has been particularly shown and described hereinabove. Rather, the scope includes both combinations and sub combinations of the various features described hereinabove, as well as variations and modifications thereof which would occur to persons skilled in the art upon reading the foregoing description and which are not disclosed in the prior art.

As described above, one aspect of the present technology is the gathering and use of physiological data to improve a user's experience of an electronic device with respect to interacting with electronic content. The present disclosure contemplates that in some instances, this gathered data may include personal information data that uniquely identifies a specific person or can be used to identify interests, traits, or tendencies of a specific person. Such personal information data can include physiological data, demographic data, location-based data, telephone numbers, email addresses, home addresses, device characteristics of personal devices, or any other personal information.

The present disclosure recognizes that the use of such personal information data, in the present technology, can be used to the benefit of users. For example, the personal information data can be used to improve interaction and control capabilities of an electronic device. Accordingly, use of such personal information data enables calculated control of the electronic device. Further, other uses for personal information data that benefit the user are also contemplated by the present disclosure.

The present disclosure further contemplates that the entities responsible for the collection, analysis, disclosure, transfer, storage, or other use of such personal information or physiological data will comply with well-established privacy policies or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

Despite the foregoing, the present disclosure also contemplates implementations in which users selectively block the use of, or access to, personal information data. That is, the present disclosure contemplates that hardware or software elements can be provided to prevent or block access to such personal information data. For example, in the case of user-tailored content delivery services, the present technology can be configured to allow users to select to “opt in” or “opt out” of participation in the collection of personal information data during registration for services. In another example, users can select not to provide personal information data for targeted content delivery services. In yet another example, users can select to not provide personal information, but permit the transfer of anonymous information for the purpose of improving the functioning of the device.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences or settings based on non-personal information data or a bare minimum amount of personal information, such as the content being requested by the device associated with a user, other non-personal information available to the content delivery services, or publicly available information.

In some embodiments, data is stored using a public/private key system that only allows the owner of the data to decrypt the stored data. In some other implementations, the data may be stored anonymously (e.g., without identifying or personal information about the user, such as a legal name, username, time and location data, or the like). In this way, other users, hackers, or third parties cannot determine the identity of the user associated with the stored data. In some implementations, a user may access his or her stored data from a user device that is different than the one used to upload the stored data. In these instances, the user may be required to provide login credentials to access their stored data.

Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter.

Unless specifically stated otherwise, it is appreciated that throughout this specification discussions utilizing the terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general purpose computing apparatus to a specialized computing apparatus implementing one or more implementations of the present subject matter. Any suitable programming, scripting, or other type of language or combinations of languages may be used to implement the teachings contained herein in software to be used in programming or configuring a computing device.

Implementations of the methods disclosed herein may be performed in the operation of such computing devices. The order of the blocks presented in the examples above can be varied for example, blocks can be re-ordered, combined, or broken into sub-blocks. Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various objects, these objects should not be limited by these terms. These terms are only used to distinguish one object from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “or” as used herein refers to and encompasses any and all possible combinations of one or more of the associated listed items. It will be further understood that the terms “comprises” or “comprising,” when used in this specification, specify the presence of stated features, integers, steps, operations, objects, or components, but do not preclude the presence or addition of one or more other features, integers, steps, operations, objects, components, or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description and summary of the invention are to be understood as being in every respect illustrative and exemplary, but not restrictive, and the scope of the invention disclosed herein is not to be determined only from the detailed description of illustrative implementations but according to the full breadth permitted by patent laws. It is to be understood that the implementations shown and described herein are only illustrative of the principles of the present invention and that various modification may be implemented by those skilled in the art without departing from the scope and spirit of the invention.

What is claimed is:

1. A method comprising:
at an electronic device having a processor and a tracking component, wherein the tracking component comprises an integrated circuit that includes a plurality of lasers and one or more photodiodes:
determining, by the tracking component, an expected position of a portion of an eye relative to the tracking component;
based on the expected position of the portion of the eye, selectively activating a subset of the plurality of lasers to project light towards the portion of the eye, wherein reflected light of the projected light are sensed via a subset of the one or more photodiodes; and
determining a current position of the portion of the eye based on coherence-based measurements using the projected light and the reflected light.

2. The method of claim 1, wherein determining the expected position of the portion of the eye relative to the tracking component is based on a previous tracked position of the eye.

3. The method of claim 1, wherein determining the expected position of the portion of the eye relative to the tracking component is based on sensor data received from one or more sensors, the sensor data corresponding to a plurality of reflections of light produced by the plurality of lasers or another light source and reflected from the eye.

4. The method of claim 1, wherein the tracking component further comprises an integrated self-mixing interferometer (SMI) array.

5. The method of claim 4, wherein the tracking component is configured to selectively switch scanning modes based on an SMI switching technique of the SMI array.

6. The method of claim 1, wherein determining the expected position of the portion of the eye relative to the tracking component is based on a first scanning mode, wherein the method further comprises:
switching to a second scanning mode, different than the first scanning mode, to scan the eye based on the determined current position and an orientation of the eye.

7. The method of claim 1, wherein selectively activating the subset of the plurality of lasers is based on a scanning mode of the tracking component.

8. The method of claim 1, wherein the coherence-based measurement comprises optical coherence tomography (OCT).

9. The method of claim 1, wherein the coherence-based measurements comprises sub-surface information associated with an features below a surface of the eye.

10. The method of claim 1, wherein the integrated circuit comprises a semiconductor material having a non-planar surface shape configured to direct the projected light from each of the plurality of lasers in a plurality of different directions.

11. The method of claim 1, wherein at least one of the one or more photodiodes are integrated in a back-side of the tracking component.

12. The method of claim 1, wherein the one or more photodiodes are integrated in a front-side of the tracking component in a complementary metal-oxide semiconductor (CMOS) application specific integrated circuit (ASIC) die.

13. The method of claim 1, wherein the one or more photodiodes are infrared (IR) photodiodes.

14. The method of claim 1, wherein the plurality of lasers are monolithic silicon light sources that are integrated on the integrated circuit.

15. The method of claim 1, wherein the plurality of lasers are vertical cavity surface-emitting lasers (VCSELs).

16. The method of claim 1, wherein the one or more photodiodes comprise a photodiode array.

17. The method of claim 1, wherein the projected light of the plurality of lasers comprises infrared (IR) light.

18. The method of claim 1, wherein the electronic device is a head-mounted device (HMD).

19. An electronic device comprising:
a frame;
a transparent substrate coupled to the frame and a waveguide, wherein the waveguide is configured to display a projected image;
a tracking component coupled to the frame and comprising an integrated circuit, wherein the integrated circuit comprises a plurality of light sources and a photodiode, wherein each light source is configured to project light towards a portion of an eye, and wherein the photodiode configured to sense reflected light of the projected light; and
a processor coupled to the tracking component, the processor configured to:
selectively activate a subset of the plurality of light sources, and
determine a current position of a portion of the eye based on coherence-based measurements using the projected light and the reflected light.

20. A non-transitory computer-readable storage medium, storing program instructions executable on a device including a tracking component and one or more processors to perform operations comprising:
determining, by the tracking component, an expected position of a portion of an eye relative to the tracking component, wherein the tracking component comprises an integrated circuit that includes a plurality of lasers and one or more photodiodes;
based on the expected position of the portion of the eye, selectively activating a subset of the plurality of lasers to project light towards the portion of the eye, wherein reflected light of the projected light are sensed via a subset of the one or more photodiodes; and
determining a current position of the portion of the eye based on coherence-based measurements using the projected light and the reflected light.
